(19) United States
(12) Patent Application Publication
(10) Pub. No.: US 2024/0041304 A1
(43) Pub. Date: Feb. 8, 2024

(54) ENDOSCOPIC DEVICE

(71) Applicant: Karl Storz SE & Co. KG, Tuttlingen (DE)

(72) Inventors: Alexander Fuchs, Tuttlingen (DE); Tobias Lottes, Helmbrechts (DE); Jochen Macht, Weißdorf (DE); Uwe Renger, Tuttlingen (DE); Daniel Ulmschneider, Tuttlingen (DE)

(73) Assignee: Karl Storz SE & Co. KG, Tuttlingen (DE)

(21) Appl. No.: 18/258,688

(22) PCT Filed: Dec. 20, 2021
(86) PCT No.: PCT/EP2021/086862

§ 371 Date: Jun. 21, 2023

(30) Foreign Application Priority Data
Dec. 22, 2020 (DE) 10 2020 134 603.4

(51) Int. Cl. A61B 1/005; A61B 1/018

(52) U.S. Cl. CPC A61B 1/0057; A61B 1/0055; A61B 1/018

(57) ABSTRACT

The disclosure relates to an endoscopic device for a particularly flexible endoscope and/or an endoscopic instrument having at least one deflectable shaft segment that has a least one distal end element and at least one proximal end element, and between the two at least one first intermediate element and at least one second intermediate element that are directly adjacent and can be deflected with respect to each other. Accordingly to the disclosure, the first intermediate element and the second intermediate element are different from each other and are integrally interconnected.

### CROSS-REFERENCE TO RELATED APPLICATIONS

This application is the U.S. national stage of PCT/EP2021/086862 filed on Dec. 20, 2021, which claims priority of German Patent Application No. DE 10 2020 134 603.4 filed on Dec. 22, 2020, the contents of which are incorporated herein.

### TECHNICAL FIELD

The disclosure relates to an endoscopic device.

### BACKGROUND

Flexible endoscopes are known which comprise a flexible shaft with deflectable shaft segments. These shaft segments comprise a plurality of elements which are formed separately from one another and which are threaded onto control trains or are connected to one another via hinges.

The object of the disclosure is in particular to make available an endoscopic device of the type in question having improved properties with regard to efficiency, in particular with regard to more efficient assembly. The object is achieved, according to the disclosure, by the features of claim 1, while advantageous configurations and developments of the disclosure can be found in the dependent claims.

### SUMMARY

The disclosure starts out from an endoscopic device having at least one deflectable shaft segment which has at least one distal end element and at least one proximal end element, and, between these, at least one first intermediate element and at least one second intermediate element which are directly adjacent and are deflectable with respect to each other.

It is proposed that the first intermediate element and the second intermediate element are designed differently from each other and are integrally connected to each other. Advantageously, assembly can be made more efficient, since the elements of the shaft segment no longer have to be individually connected to one another in a form-fitting and/or force-fitting manner. Rather, such a shaft segment can be produced in one piece, preferably in one step, for example by injection molding, 3D printing, laser processing or the like.

As used herein, the term “endoscopic device” should be understood in particular as meaning a constituent part, preferably a functional constituent part, in particular a subassembly and/or a structural component and/or functional component of an endoscopic instrument and/or of an endoscope, in particular a flexible endoscope. Alternatively, the endoscopic device can at least partly, preferably at least to a large extent and particularly preferably completely embody an endoscope and/or an endoscopic instrument. The endoscope is in particular a flexible endoscope. The endoscope is particularly preferably designed as a disposable endoscope or single-use endoscope, which is configured for one-time use only. In particular, such an endoscope can no longer be cleaned after use and/or has a specific defect after use, so that it cannot be reused. For example, such an endoscope can consist of plastics that are unable to withstand the temperatures required for treatment in an autoclaving process. “Endoscopically” should be understood in particular as also meaning minimally invasive. The expression “at least to a large extent” should be understood as meaning in particular at least 55%, preferably at least 65%, preferably at least 75%, particularly preferably at least 85%, and very particularly preferably at least 95%, or advantageously completely, to be precise in relation to a volume and/or mass of an object, in particular. The endoscopic device is, for example, configured to be introduced, at least partly and preferably to at least a large extent, into an orifice, in particular an artificial and/or natural orifice, in particular a body orifice, in order to perform a treatment and/or an examination there. An endoscopic instrument can, for example, be in the form of endoscopic forceps, endoscopic scissors, an endoscopic scalpel, an endoscopic stapler or the like. In particular, “configured” should be understood as meaning specifically programmed, provided, designed and/or equipped. An object being configured for a specific function should be understood as meaning in particular that the object satisfies and/or carries out this specific function in at least one application state and/or operational state. Furthermore, the endoscopic device can be part of a surgical system. A surgical system should be understood in particular as a system configured for performing a surgical procedure, for example an endoscopic and/or minimally invasive procedure, which system comprises at least one surgical robot. The surgical robot can comprise at least one surgical robot arm or a plurality of surgical robot arms. The endoscopic device can be controllable and/or actuatable by the surgical robot, in particular the surgical robot arm. The endoscopic device can be able to be coupled releasably to the surgical robot, for example in order to permit exchange and/or cleaning of the endoscopic device. Moreover, the surgical system can comprise at least one controller, which is configured for manual and/or automated control of the surgical robot.

The endoscopic device has in particular at least one shaft which can be inserted at least partially and preferably at least to a large extent into an in particular artificial and/or natural opening, in particular a body orifice. The shaft is preferably flexible. The shaft comprises, for example, at least one end portion and/or a further end portion, the end portion being a distal end portion and/or the further end portion being a proximal end portion, for example. “Distal” should be understood in particular to mean facing a patient and/or distant from a user during operation. “Proximal” should be understood in particular to mean distant from a patient and/or facing a user during operation. The shaft has for example an axis of principal extent. An axis of principal extent of an object should be understood as an axis which runs through the geometric midpoint and/or center of gravity of the object and is at least substantially parallel to a direction of principal extent of the object. Here, a “direction of principal extent” of an object should be understood in particular to mean a direction that extends parallel to a longest edge of a smallest imagined cuboid that just still completely surrounds the object. A longitudinal extent for example of the shaft is identical to the direction of principal extent of the latter. Here, “at least substantially parallel” should be understood in particular as an orientation of a direction relative to a reference direction, in particular in a plane, wherein the direction and the reference direction enclose an angle of 0° in particular in consideration of a maximum deviation of less than 8°, advantageously of less than ° and particularly advantageously of less than 2°. A width can be measured at least substantially perpendicular to the longitudinal extent. Here, “at least substantially perpendicular” should be understood in particular as an orientation of a direction relative to a reference direction, in particular in a plane, wherein the direction and the reference direction enclose an angle of 90°, in particular in consideration of a maximum deviation of less than 8°, advantageously of less than ° and particularly advantageously of less than 2°.

The endoscopic device can have a plurality of first intermediate elements and a plurality of second intermediate elements that can be formed at least substantially identical to one another. “At least substantially identical” should be understood as meaning either identical or identical apart from assembly and/or production tolerances. The first connection elements and the second connection elements can be arranged alternately in line.

The fact that “an object and a further object have an at least partly integral embodiment/connection” should be understood as meaning in particular that at least one element and/or part of the object and at least one element and/or part of the further object have an integral embodiment/connection. In particular, “integral” should be understood to mean at least cohesively bonded, for example by a welding process, an adhesive bonding process, a spraying process and/or any other process appearing expedient to a person skilled in the art. Furthermore, “integral” can also be understood as meaning in one piece. “In one piece” should be understood in particular as meaning formed in one piece, for example by production from one cast and/or by production in a single-component or multiple-component injection method and, advantageously, from a single blank. Components of the endoscopic device should be connected to one another at least partially by form-fitting and/or force-fitting engagement.

A “force-fitting and/or form-fitting engagement” should be understood in particular as meaning connected, preferably releasably connected, wherein a holding force is transmitted between two objects preferably by geometric interlocking of the structural components in one another and/or by a frictional force that preferably acts between the objects. Alternatively or in addition, components of the endoscopic device can be connected to one another by cohesive bonding. “Cohesive bonding” should be understood in particular as meaning that the objects are held together by atomic or molecular forces, for example by soldering, welding, adhesion and/or vulcanization.

The shaft can have a deflectable portion. The endoscopic device can have at least one deflection mechanism for deflecting the deflectable portion. The deflection mechanism is designed in particular for mechanical deflection of the deflectable shaft segment. The deflectable shaft segment is formed at least partially, preferably at least to a large extent and particularly preferably completely from a plastic, preferably a flexible and/or elastic plastic. In particular, the deflectable shaft segment can be deflected in at least one plane. In particular, the deflection mechanism comprises at least one control train, which is preferably designed to be flexurally slack. A “flexurally slack component” should be understood in particular as a component, preferably an elongate component, which has flexurally slack properties at least in one direction perpendicular to a direction of principal extent. It should preferably be understood in particular as a dimensionally non-stable component. Particularly preferably, it should be understood in particular as a component which, in an elongated state, applies a counterforce to a pressure force acting parallel to a direction of principal extent, which counterforce is less than a weight force of the component. Preferably, the counterforce is at most 70%, preferably at most 50% and particularly preferably at most 30% of a weight force. Here, an “elongate component” should be understood in particular as a component having a transverse extent that is many times smaller than a longitudinal extent. Here, “many times smaller” should be understood in particular as at least 3 times smaller, preferably at least 5 times smaller and particularly preferably at least 10 times smaller.

It is further proposed that the deflectable shaft segment comprises at least two mutually opposite webs which integrally connect the first intermediate element and the second intermediate element and which define an axis of rotation about which the first intermediate element and the second intermediate element can be deflected upon deflection of the deflectable segment. Stability can advantageously be improved and, at the same time, sufficient deflectability of the deflectable shaft segment can be ensured. Furthermore, the deflectable shaft segment can have at least two webs which connect the first intermediate element and the second intermediate element to each other. The two webs are preferably arranged opposite each other. The two webs and their deflection points define in particular the plane from which the deflectable shaft segment can be deflected. It is conceivable that the web consists of a different material than the intermediate elements. For example, the web could be injection molded onto the intermediate elements. However, the web preferably consists of the same material as the intermediate elements.

In a further aspect of the disclosure, which can in particular be considered separately from or in combination with the preceding aspects, it is further proposed that the first intermediate element and the second intermediate element each have a receiving recess which is closed along a circumference of the intermediate elements and in which at least one working channel and/or at least one conductor strand can be arranged. Assembly can advantageously be further simplified since the working channel and electronic conductors can be inserted in one work step. In particular, the endoscopic device comprises the working channel and/or the electronic conductor. The electronic conductor can be, for example, a flexprint cable or the like. The working channel is in particular formed by a tube, in particular a flexible tube. The intermediate segments preferably each have only a single receiving recess in which all the electronic conductors or working channels are arranged.

It is further proposed that, in a non-deflected state of the deflectable shaft segment, the receiving recesses of the intermediate elements are arranged congruently to each other. Introduction of the working channel and/or of the electrical conductors can advantageously be further improved.

It is further proposed that the receiving recess has a main axis, along which the receiving recess is mirror-symmetrical, and comprises a secondary axis, which is oriented at least substantially perpendicular to the main axis and along which the receiving recess is not mirror-symmetrical. A secure seat of the working channel and of the electrical conductors in the deflectable shaft segment can advantageously be ensured. A main axis is to be understood in particular as the axis along the direction of principal extent of the receiving recess. A “main axis” is to be understood in particular as meaning the axis which runs along the greatest diameter of the receiving recess. A “secondary axis” is to be understood in particular as meaning the axis which runs along the smallest diameter of the receiving recess. The main axis of the receiving recess is in particular at least substantially perpendicular to a principal extent of the shaft. The receiving recess has in particular an oval shape and is particularly preferably in the shape of a line or curve of an egg. In particular, the receiving recess has the shape of two straight lines which are in tangential contact with each other via these circles and which connect these circles, one of the circles having a larger radius than the other. If the working channel or the tube forming the working channel is inserted into the receiving recess, it encompasses the working channel by a circumferential angle of more than 180°.

It is further proposed that the receiving recess and the webs are oriented with respect to one another such that, in a non-deflected state of the deflectable shaft segment, the main axis of the receiving recess is at least substantially parallel to the axis of rotation of the webs. Advantageously, a deflectability of the deflectable shaft segment can be improved.

It is additionally proposed that the first intermediate element and the second intermediate element each have at least two guide hooks which lie opposite each other and in which at least one control train for deflecting the deflectable shaft segment can be arranged. Assembly can be further simplified, since the intermediate elements do not have to be threaded onto the control train as is customary, and instead the control train can be inserted laterally into the guide hooks. The two guide hooks lie mirror-symmetrically opposite each other relative to the main axis.

It is further proposed that the guide hooks each have an insertion opening, the insertion opening of the first intermediate element and the insertion opening of the second intermediate element opening out in opposite directions. Assembly can be further simplified, since the control train can be woven into the shaft segment. “Laterally” should also be understood here in particular as radially.

It is further proposed that the guide hooks are arranged offset with respect to the web along a circumference of the intermediate elements. A situation can be advantageously avoided in which the pivot point, which is defined by the webs, is reinforced or blocked by a control train. In particular, the guide hooks and the webs are offset from one another by at least 45°, but preferably by 90°, along the circumference of a respective intermediate element.

It is further proposed that a camera head can be arranged in the distal end element. Advantageously, assembly can be further simplified since a structural unit consisting of the camera head and of the deflectable shaft segment can be created in this way, which can be installed in a further step or can be replaced as a whole during repair and/or maintenance. The camera head is in particular connected to the end element in a force-fitting and/or form-fitting manner. Alternatively or additionally, the camera head can be integrally connected to the end element. It would also be conceivable that an end effector could be arranged on the distal end element, for example forceps, scissors or the like. It is further proposed that the proximal end element can be connected at least in a force-fitting and/or form-fitting manner to a further shaft segment.

### BRIEF DESCRIPTION OF DRAWINGS

Further advantages will become clear from the following description of the drawings. The drawings show an exemplary embodiment of the disclosure. The drawings, the description and the claims contain numerous features in combination. A person skilled in the art will expediently also consider the features individually and combine them into further meaningful combinations.

FIG. 1 shows a schematic perspective view of an endoscope with an endoscopic device,

FIG. 2 shows a schematic perspective view of a deflectable shaft of the endoscopic device,

FIG. 3 shows a schematic sectional view of an intermediate element of a deflectable shaft segment of the deflectable shaft, and

FIG. 4 shows a schematic sectional view of an intermediate element of a deflectable shaft segment of an alternative endoscopic device.

FIG. 5 shows a schematic sectional view of an intermediate element of a deflectable shaft segment of a further alternative endoscopic device.

### DETAILED DESCRIPTION OF PREFERRED EMBODIMENTS

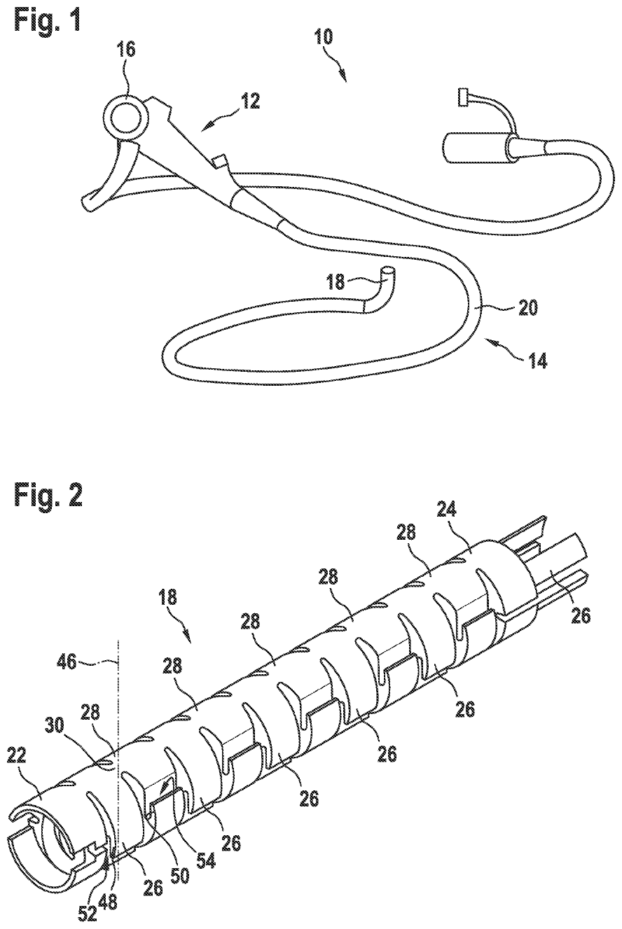

FIG. 1 shows a schematic perspective view of an endoscope 10 with an endoscopic device. In the present case, the endoscope 10 is designed as a flexible endoscope. Furthermore, the endoscope 10 is designed as a disposable endoscope or single-use endoscope. In the present case, the endoscopic device forms the endoscope 10 completely. Alternatively, the endoscopic device could also be just part of an endoscope. It is conceivable that, instead of forming an endoscope, the endoscopic device could also form an endoscopic instrument, such as endoscope forceps, scissors or the like.

The endoscopic device comprises a handle 12. The endoscopic device moreover comprises a shaft 14. The shaft 14 is flexible. The handle 12 comprises a control means 16. In the present case, the control means 16 is in the form of a lever. A deflection of at least part of the shaft can be achieved by means of the control means. The shaft 14 has a deflectable shaft segment 18. Furthermore, the shaft 14 has a further shaft segment 20. The further shaft segment 20 is a passive or flexible shaft segment 20, which adapts its bending to external conditions. By contrast, the deflectable shaft segment 18 is actively controllable or deflectable. The deflectable shaft segment 18 and the further shaft segment 20 are connected to each other.

FIG. 2 shows a schematic perspective view of the deflectable shaft segment 18. The deflectable shaft segment 18 is formed in one piece. For example, the deflectable shaft segment 18 can produced by injection molding. The deflectable shaft segment 18 has a plurality of interconnected elements 22, 24, 26, 28 movable relative to one another. One of these elements 22, 24, 26, 28 is a distal end element 22. The endoscopic device has a camera head (not shown). The camera head is connected to the distal end element 22. In the present case, the camera head is connected to the distal end element 22 in a force-fitting and/or form-fitting manner. As an alternative or in addition, the camera head can also be connected to the distal end element in a materially bonded manner. Alternatively, instead of a camera head, an end effector can also be arranged on the distal end element, for example if the endoscopic device is part of an endoscopic instrument or forms the latter.

Furthermore, the deflectable shaft segment has a proximal end element 24. The proximal end element 24 is connected to the other shaft segment 20 in a force-fitting and/or form-fitting manner. The proximal end element 24 has a plurality of tongues, which serve to produce the connection.

Furthermore, the deflectable shaft segment 18 has a plurality of intermediate elements 26, 28. In the present case, the deflectable shaft segment 18 has a number of intermediate elements 26, 28, for example ten of them. The number of intermediate elements 26, 28 can be freely chosen by a person skilled in the art depending on a desired deflection angle or deflection radius of the deflectable shaft segment 18. In the present case, the deflectable shaft segment 18 has different intermediate elements 26, 28, specifically first intermediate elements 26 and second intermediate elements 28. A first intermediate element 26 and a second intermediate element 28 are arranged adjacent to each other. Except at the proximal and distal ends of the deflectable shaft segment, which are closed off by the proximal and distal end elements 22, 24, a first intermediate element 26 is adjoined directly by two second intermediate elements 28. Furthermore, every second intermediate element 28 is adjoined directly by two first intermediate elements 26. In other words, the first intermediate elements 26 and the second intermediate elements 28 are arranged or lined up alternately. Since the first intermediate elements 26 do not differ from one another, only one of them is explained in more detail in the present description and provided with the same reference number. Since the second intermediate elements 28 do not differ from one another either, only one second intermediate element is explained in more detail in the present description and provided with the same reference number.

The first intermediate element 26 and the second intermediate element 28 are designed differently from each other. The first intermediate element 26 and the second intermediate element 28 are directly adjacent. The first intermediate element 26 and the second intermediate element 28 are deflectable relative to each other. The first intermediate element 26 and the second intermediate element 28 are designed differently from each other and are connected to each other in one piece.

The deflectable shaft segment 18 has two webs 30, which connect the first intermediate element 26 and the second intermediate element 28 to each other. The webs 30 are arranged opposite each other. The webs 30 define an axis of rotation 46 about which the intermediate elements 26, 28 are deflected relative to each other.

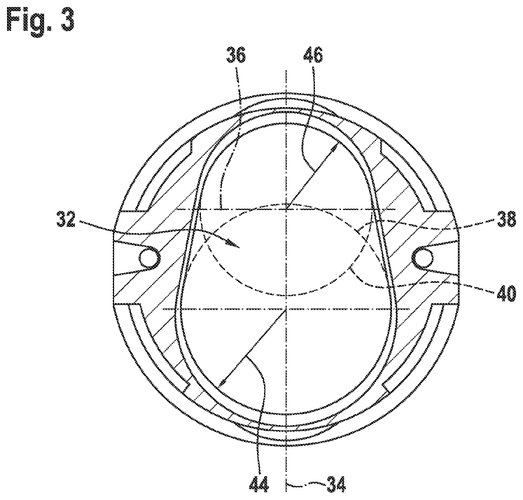

FIG. 3 shows a schematic sectional view of the deflectable shaft segment 18. The first intermediate element 26 and the second intermediate element 28 each have a receiving recess 32 which is closed along a circumference of the intermediate elements 26, 28 and in which at least one working channel (not shown) and/or at least one conductor strand (not shown) can be arranged. In a non-deflected state of the deflectable shaft segment 18, the receiving recesses 32 of the intermediate elements 26, 28 are arranged congruently to each other. The endoscopic device also comprises the working channel and/or the conductor strand. The working channel is formed by a tube. The conductor strand is a flexprint cable. For the sake of clarity, this and the working channel are not shown in the figures.

The receiving recess 32 has a main axis 34. The receiving recess 32 is mirror-symmetrical along the main axis 34. Furthermore, the receiving recess 32 has a secondary axis 36. The secondary axis 36 is at least substantially perpendicular to the main axis 34. The receiving recess 32 is not mirror-symmetrical along the secondary axis 36. The receiving recess 32 has an oval shape. To be more precise, the receiving recess 32 has the shape of a line or curve of an egg. The receiving recess 32 has the shape of two circles 38, 40 which are connected to each other via two straight lines which tangentially adjoin these circles 38, 40 and are tangent to these circles 38, 40. One circle 38 has a radius 42, which is greater than a radius 44 of the other circle 40.

The working channel can be arranged in the region of the circle 38 having the greater radius 42 of the receiving recess 32. If the tube forming the working channel lies in the receiving recess 32, the circle 38 encompasses the working channel by a circumferential angle of more than 180°, as a result of which the working channel is advantageously secured against slipping out. The conductor strand is arranged in the region of the circle 40 of smaller radius 44.

The receiving recess 32 and the webs 30 are oriented relative to each other such that, in a non-deflected state of the deflectable shaft segment 18, the axis of rotation 46 defined by the pivot points of the webs 30 is at least substantially parallel to the main axis of the receiving recess 32.

The endoscopic device has a deflection mechanism for power transmission. The deflection mechanism comprises at least one control train. For the sake of clarity, the deflection mechanism and the control train are not shown in the figures. In the present case, the deflection mechanism comprises two control trains. These control trains are coupled to the control means 16. The control trains are triggered by means of the control means 16. The control trains are arranged on the deflectable shaft segment 18. Furthermore, the control trains are connected to the distal end element 22 of the deflectable shaft segment 18.

For one arrangement of the control train, the first intermediate element 26 and the second intermediate element 28 each have at least one guide hook 48, 50. The control train for deflecting the deflectable shaft segment 18 can be arranged in the guide hooks 48, 50. In the present case, each of the intermediate elements 26, 28 has two guide hooks 48, 50, respectively. The guide hooks 48, 50 are arranged offset with respect to the web along a circumference of the intermediate elements 26, 28. The guide hooks 48, 50 are arranged opposite each other.

The guide hooks 48, 50 each have an insertion opening 52, 54. The insertion opening 52 of the first intermediate element 26 and the insertion opening 54 of the second intermediate element 28 are arranged oppositely. Since the intermediate elements 26, 28 are arranged alternately in line, the respective direction of opening of the insertion openings 52, 54 of the guide hooks 48, 50 also alternates.

In the same way as the intermediate elements 26, 28 described above, the distal and the proximal end element 22, 24 also each have a receiving recess 32. Furthermore, the distal and the proximal end element can have guide hooks in the same way. The distal end element 22 is likewise integrally connected to one of the first intermediate elements 26 via webs 30. Furthermore, the proximal end element 24 is connected in one piece to one of the second intermediate elements 28 in the same way.

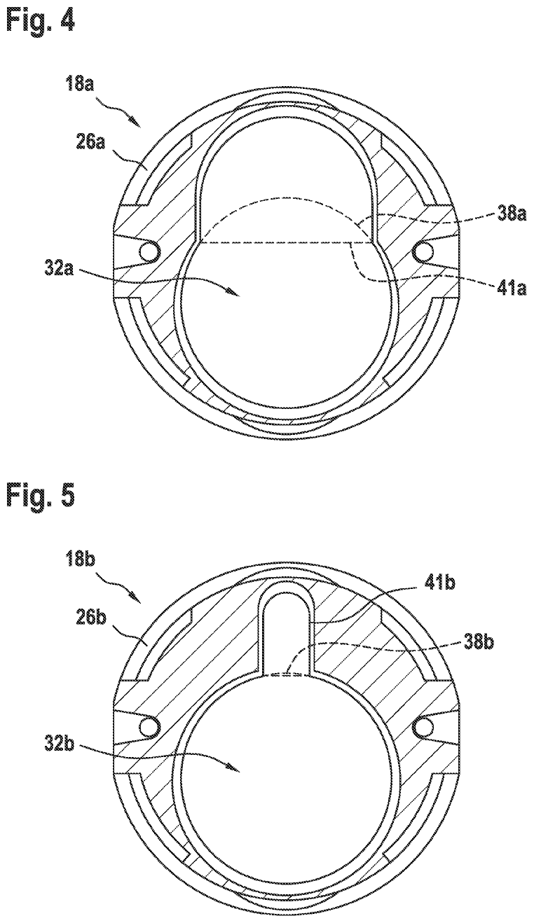

FIGS. 4 and 5 show two further exemplary embodiments of the disclosure. The following descriptions and the drawings are limited essentially to the differences between the exemplary embodiments, wherein, in respect of components with the same label, in particular in respect of components with the same reference signs, reference is also made, as a matter of principle, to the drawings and/or the description of the other exemplary embodiments. In order to distinguish between the exemplary embodiments, the letters a and b have been appended to the reference signs of the exemplary embodiments that follow.

FIG. 4 shows a sectional view of an intermediate element 26a of a deflectable shaft segment 18a of an alternative endoscopic device. The intermediate element 26a has a receiving recess 32a. The receiving recess 32a has a main axis 34a. The receiving recess 32a is mirror-symmetrical along the main axis 34a. Furthermore, the receiving recess 32a has a secondary axis 36a. The secondary axis 36a is at least substantially perpendicular to the main axis 34a. The receiving recess 32a is not mirror-symmetrical along the secondary axis 36a. The receiving recess 32a has a keyhole-like shape. The receiving recess 32a has a shape of a circle 38a which is widened with an archway 41a. The circle 38a delimits an area that is larger than an area delimited by the archway 41a.

A working channel can be arranged in the region of the circle 38a. If a tube forming the working channel lies in the receiving recess 32a, the circle 38a encompasses the working channel by a circumferential angle of more than 270°, as a result of which the working channel is advantageously secured against slipping out. A conductor strand is arranged in the region of the archway 41a.

FIG. 5 shows a sectional view of an intermediate element 26b of a deflectable shaft segment 18b of a further alternative endoscopic device. The intermediate element 26a has a receiving recess 32b. The receiving recess 32b has a shape of a circle 38b which is widened with an archway 41b. The circle 38b delimits an area which is substantially larger than an area delimited by the archway 41b. In the present case, the area delimited by the circle 38b is larger, at least by the factor 5 and preferably by the factor 10, than the area delimited by the archway. The archway 41b is narrowed to a slit in the present case.

A working channel can be arranged in the region of the circle 38b. If a tube forming the working channel lies in the receiving recess 32b, the circle 38b encompasses the working channel by a circumferential angle of more than 340°, as a result of which the working channel is advantageously secured against slipping out. A conductor strand is arranged in the region of the archway 41b.

1. An endoscopic device for a flexible endoscope and/or an endoscopic instrument, the endoscopic device comprising:
at least one deflectable shaft segment having a distal end element and a proximal end element,
at least one first intermediate element and at least one second intermediate element disposed between the distal end and the proximal end the at least one first intermediate element and the at least one second intermediate element are directly adjacent and are deflectable with respect to each other;
wherein the first intermediate element and the second intermediate element each have a receiving recess which is closed along a circumference of the at least one first and the at least one second intermediate elements and in which at least one working channel and at least one conductor strand can be arranged and wherein the receiving recess has a keyhole-like shape.

2. The endoscopic device as forth in claim 1, wherein the deflectable shaft segment includes at least two webs lying opposite each other, which integrally connect the at least one first intermediate element and the second intermediate element, wherein the at least two webs define an axis of rotation about which the first intermediate element and the second intermediate element are deflectable upon deflection of the deflectable shaft segment.

3. The endoscopic device as set forth in claim 1, wherein the first intermediate element and the second intermediate element are designed differently from each other and are integrally connected to each other.

4. The endoscopic device as set forth in claim 1, wherein, in a non-deflected state of the deflectable shaft segment, the receiving recesses of the at least one first and at least one second intermediate elements are arranged congruently to each other.

5. The endoscopic device as set forth in claim 1, wherein the receiving recess has a main axis, along which the receiving recess is mirror-symmetrical, and secondary axis, which is oriented at least substantially perpendicular to the main axis and along which the receiving recess is not mirror-symmetrical.

6. The endoscopic device as set forth in claim 5, wherein the receiving recess and the at least two webs are oriented with respect to one another such that, in a non-deflected state of the deflectable shaft segment, the main axis of the receiving recess is at least substantially parallel to the axis of rotation of the at least two webs.

7. The endoscopic device as set forth in claim 1, the at least one first intermediate element and the at least one second intermediate element each have at least two guide hooks which lie opposite each other and in which at least one control train for deflecting the deflectable shaft segment can be arranged.

8. The endoscopic device as set forth in claim 7, wherein the guide hooks have an insertion opening, the insertion opening of the at least one first intermediate element and the insertion opening of the at least one second intermediate element opening out in opposite directions.

9. The endoscopic device as set forth in claim 2, wherein the guide hooks are arranged offset with respect to the at least two webs along a circumference of the at least one first and the at least one second intermediate elements.

10. The endoscopic device as set forth in claim 1, wherein a camera head can be arranged on the distal end element.

11. The endoscopic device as in set forth in claim 1, wherein the proximal end element can be connected to a further shaft segment at least in a force-fitting and/or form-fitting manner.

12. An endoscope, in particular a flexible endoscope, and/or endoscopic instrument having at least one endoscopic device as set forth in claim 1.
